(19) United States
(12) Patent Application Publication
(10) Pub. No.: US 2024/0428941 A1
(43) Pub. Date: Dec. 26, 2024

(54) Multimodal Artificial Intelligence Assistant for Health Care

(71) Applicant: ALFREDAI, INC., Austin, TX (US)

(72) Inventor: Ajay Tripuraneni, Austin, TX (US)

(21) Appl. No.: 18/749,405

(22) Filed: Jun. 20, 2024

Related U.S. Application Data
(60) Provisional application No. 63/522,040, filed on Jun. 20, 2023.

(51) Int. Cl. G16H 50/20; G16H 10/60

(52) U.S. Cl. CPC G16H 50/20; G16H 10/60

(57) ABSTRACT

Devices, systems, and methods for providing a patient with a personalized digital healthcare assistant. A medical history and biometric information for a patient are received by a computing device. A text conversation is conducted with the patient using a large language model (LLM) through a user interface (UI) to obtain a plurality of responses. The medical history, the plurality of responses, and the biometric information are stored in the non-transitory computer-readable memory medium. An intervention proposal is determined using a machine learning software based at least in part on the medical history, the plurality of responses, and the biometric information, and the intervention proposal is displayed on a display.

### PRIORITY INFORMATION

This application claims benefit and priority to U.S. Provisional Application No. 63/522,040, entitled “Atrial Fibrillation Patient Experience Platform,” and filed Jun. 20, 2023, which is hereby incorporated by reference in its entirety as though fully and completely set forth herein.

### FIELD

The present application relates to systems and methods to provide personalized healthcare support for a patient.

### DESCRIPTION OF THE RELATED ART

Medical care can be expensive in the United States and throughout the world, and in many cases patients struggle to navigate a complex and confusing healthcare system to acquire adequate care for their health. To treat a complex condition such as atrial fibrillation (AF), or another health problem, a patient may separately interface with a large number of entities, such as their physician, pharmacist, patient call center, and hospital. Navigating the health care system to have their condition treated effectively may incur a large amount of time and personal expense. Accordingly, improvements in the field of providing health care to patients are desired.

### SUMMARY

Embodiments relate to devices, systems, and methods to provide a patient with a personalized digital healthcare assistant. In some embodiments, a non-transitory computer-readable memory medium stores program instructions that are executable by a processor to cause a computing device to provide the personalized digital healthcare assistance.

In some embodiments, a medical history for a patient is received. In some embodiments, a text conversation is conducted with the patient using a large language model (LLM) through a user interface (UI) to obtain a plurality of responses.

In some embodiments, biometric information for the patient is received from at least one biometric device.

In some embodiments, the medical history, the plurality of responses, and the biometric information are stored in the non-transitory computer-readable memory medium.

In some embodiments, an intervention proposal is determined using a machine learning software based at least in part on the medical history, the plurality of responses, and the biometric information.

In some embodiments, the intervention proposal is displayed on a display.

The techniques described herein may be implemented in and/or used with a number of different types of devices, including but not limited to computers, cellular phones, tablet computers, wearable computing devices, portable media players, and any of various other computing devices.

This Summary is intended to provide a brief overview of some of the subject matter described in this document. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

A better understanding of the present subject matter can be obtained when the following detailed description of various embodiments is considered in conjunction with the following drawings, in which:

FIG. 1 is a computer system block diagram, according to some embodiments;

FIG. 2 is a flow diagram illustrating operation of a multimodal artificial intelligence (AI) assistant for a patient to manage their own health, according to some embodiments;

FIG. 3 is a flowchart illustrating a method for providing a health intervention proposal, according to some embodiments;

FIG. 4 is a diagram illustrating factors that may contribute to social determinants of health, according to some embodiments;

FIG. 5 is a diagram illustrating examples of clinical and non-clinical health goals, according to some embodiments;

FIG. 6 is a flowchart illustrating the generation of personalized action items for a patient, according to some embodiments;

FIG. 7 is a flowchart illustrating a user interface for categorizing patient archetypes and advising patient behavior, according to some embodiments;

FIG. 8 is a diagram illustrating the determination of a possible behavioral nudge based on a root behavior and a patient's perspective, according to some embodiments;

FIG. 9 is a diagram illustrating different computational modules for providing health services to a patient, according to some embodiments;

FIG. 10 is a diagram illustrating data collection and AI bot operation for providing health services to a patient, according to some embodiments;

FIG. 11 contrasts the provision of healthcare to a patient with and without a multimodal AI assistant, according to some embodiments;

FIG. 12 is a flowchart showing a computational flow for providing expert health monitoring for a patient, according to some embodiments;

FIG. 13 is a flowchart illustrating a method for a medical history bot to construct a medical history for a patient with atrial fibrillation (AF), according to some embodiments; and

FIG. 14 is a flowchart illustrating a workflow for optimizing risk factors and providing behavioral nudges to a patient, according to some embodiments.

While the features described herein may be susceptible to various modifications and alternative forms, specific embodiments thereof are shown by way of example in the drawings and are herein described in detail. It should be understood, however, that the drawings and detailed description thereto are not intended to be limiting to the particular form disclosed, but on the contrary, the intention is to cover all modifications, equivalents and alternatives falling within the spirit and scope of the subject matter as defined by the appended claims.

### DETAILED DESCRIPTION

### Terms

The following is a glossary of terms used in this disclosure:

Memory Medium—Any of various types of non-transitory memory devices or storage devices. The term “memory medium” is intended to include an installation medium, e.g., a CD-ROM, floppy disks, or tape device; a computer system memory or random access memory such as DRAM, DDR RAM, SRAM, EDO RAM, Rambus RAM, etc.; a non-volatile memory such as a Flash, magnetic media, e.g., a hard drive, or optical storage; registers, or other similar types of memory elements, etc. The memory medium may include other types of non-transitory memory as well or combinations thereof. In addition, the memory medium may be located in a first computer system in which the programs are executed, or may be located in a second different computer system which connects to the first computer system over a network, such as the Internet. In the latter instance, the second computer system may provide program instructions to the first computer for execution. The term “memory medium” may include two or more memory mediums which may reside in different locations, e.g., in different computer systems that are connected over a network. The memory medium may store program instructions (e.g., embodied as computer programs) that may be executed by one or more processors.

Portable Memory Device—Any of various types of physical media containing a memory medium, wherein the portable memory device is configured to communicate with a computing device to receive and transmit data from the memory medium. Examples of portable memory devices include universal serial bus (USB) drives, or “thumb drives”, portable hard drives, and other types of portable memory media.

Carrier Medium—a memory medium as described above, as well as a physical transmission medium, such as a bus, network, and/or other physical transmission medium that conveys signals such as electrical, electromagnetic, or digital signals.

Processing Element—refers to various elements or combinations of elements that are capable of performing a function in a device, such as a user equipment or a cellular network device. Processing elements may include, for example: processors and associated memory, portions or circuits of individual processor cores, entire processor cores, processor arrays, circuits such as an ASIC (Application Specific Integrated Circuit), programmable hardware elements such as a field programmable gate array (FPGA), as well any of various combinations of the above.

Software Program—the term “software program” is intended to have the full breadth of its ordinary meaning, and includes any type of program instructions, code, script and/or data, or combinations thereof, that may be stored in a memory medium and executed by a processor. Exemplary software programs include programs written in text-based programming languages, such as C, C++, PASCAL, FORTRAN, COBOL, JAVA, assembly language, etc.; graphical programs (programs written in graphical programming languages); assembly language programs; programs that have been compiled to machine language; scripts; and other types of executable software. A software program may comprise two or more software programs that interoperate in some manner. Note that various embodiments described herein may be implemented by a computer or software program. A software program may be stored as program instructions on a memory medium.

Hardware Configuration Program—a program, e.g., a netlist or bit file, that can be used to program or configure a programmable hardware element.

Program—the term “program” is intended to have the full breadth of its ordinary meaning. The term “program” includes 1) a software program or application which may be stored in a memory and is executable by a processor or 2) a hardware configuration program useable for configuring a programmable hardware element.

Computer System—any of various types of computing or processing systems, including a personal computer system (PC), mainframe computer system, workstation, network appliance, Internet appliance, personal digital assistant (PDA), television system, grid computing system, or other device or combinations of devices. In general, the term “computer system” can be broadly defined to encompass any device (or combination of devices) having at least one processor that executes instructions from a memory medium.

User Equipment (UE) (or “UE Device”)—any of various types of computer systems devices which are mobile or portable and which performs wireless communications. Examples of UE devices include mobile telephones or smart phones (e.g., iPhone™, Android™-based phones), portable gaming devices (e.g., Nintendo DS™, PlayStation Portable™, Gameboy Advance™, iPhone™), laptops, wearable devices (e.g. smart watch, smart glasses), PDAs, portable Internet devices, music players, data storage devices, or other handheld devices, etc. In general, the term “UE” or “UE device” can be broadly defined to encompass any electronic, computing, and/or telecommunications device (or combination of devices) which is easily transported by a user and capable of wireless communication. A UE device may be configured to communicate according to various wireless access technologies, including but not limited to cellular communications, Wi-Fi or wireless local area network WLAN communications, short-range wireless access technologies such as Bluetooth, global positioning satellite (GPS) or other global navigational satellite technologies, among other possibilities.

Measurement Device—includes instruments, data acquisition devices, smart sensors, and any of various types of devices that are configured to acquire and/or store data. A measurement device may also optionally be further configured to analyze or process the acquired or stored data. Examples of a measurement device include an instrument, such as a traditional stand-alone “box” instrument, a computer-based instrument (instrument on a card) or external instrument, a data acquisition card, a device external to a computer that operates similarly to a data acquisition card, a smart sensor, one or more DAQ or measurement cards or modules in a chassis. The measurement device may be equipped with one or more sensors for performing electromyographic measurements on a human subject to measure muscle activity, in some embodiments.

Automatically—refers to an action or operation performed by a computer system (e.g., software executed by the computer system) or device (e.g., circuitry, programmable hardware elements, ASICs, etc.), without user input directly specifying or performing the action or operation. Thus the term “automatically” is in contrast to an operation being manually performed or specified by the user, where the user provides input to directly perform the operation. An automatic procedure may be initiated by input provided by the user, but the subsequent actions that are performed “automatically” are not specified by the user, i.e., are not performed “manually”, where the user specifies each action to perform. For example, a user filling out an electronic form by selecting each field and providing input specifying information (e.g., by typing information, selecting check boxes, radio selections, etc.) is filling out the form manually, even though the computer system must update the form in response to the user actions. The form may be automatically filled out by the computer system where the computer system (e.g., software executing on the computer system) analyzes the fields of the form and fills in the form without any user input specifying the answers to the fields. As indicated above, the user may invoke the automatic filling of the form, but is not involved in the actual filling of the form (e.g., the user is not manually specifying answers to fields but rather they are being automatically completed). The present specification provides various examples of operations being automatically performed in response to actions the user has taken.

Approximately—refers to a value that is almost correct or exact. For example, approximately may refer to a value that is within 1 to 10 percent of the exact (or desired) value. It should be noted, however, that the actual threshold value (or tolerance) may be application dependent. For example, in some embodiments, “approximately” may mean within 0.1% of some specified or desired value, while in various other embodiments, the threshold may be, for example, 2%, 3%, 5%, and so forth, as desired or as required by the particular application.

Concurrent—refers to parallel execution or performance, where tasks, processes, or programs are performed in an at least partially overlapping manner. For example, concurrency may be implemented using “strong” or strict parallelism, where tasks are performed (at least partially) in parallel on respective computational elements, or using “weak parallelism”, where the tasks are performed in an interleaved manner, e.g., by time multiplexing of execution threads.

Various components may be described as “configured to” perform a task or tasks. In such contexts, “configured to” is a broad recitation generally meaning “having structure that” performs the task or tasks during operation. As such, the component can be configured to perform the task even when the component is not currently performing that task (e.g., a set of electrical conductors may be configured to electrically connect a module to another module, even when the two modules are not connected). In some contexts, “configured to” may be a broad recitation of structure generally meaning “having circuitry that” performs the task or tasks during operation. As such, the component can be configured to perform the task even when the component is not currently on. In general, the circuitry that forms the structure corresponding to “configured to” may include hardware circuits.

Various components may be described as performing a task or tasks, for convenience in the description. Such descriptions should be interpreted as including the phrase “configured to.” Reciting a component that is configured to perform one or more tasks is expressly intended not to invoke 35 U.S.C. § 112 (f) interpretation for that component.

### DETAILED DESCRIPTION

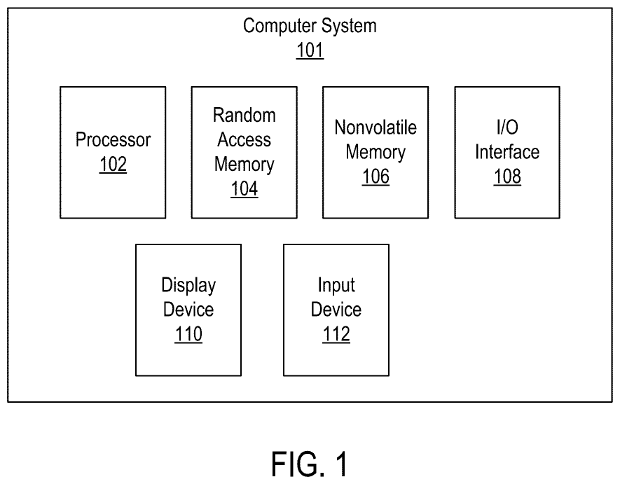

### FIG. 1—Computer System Block Diagram

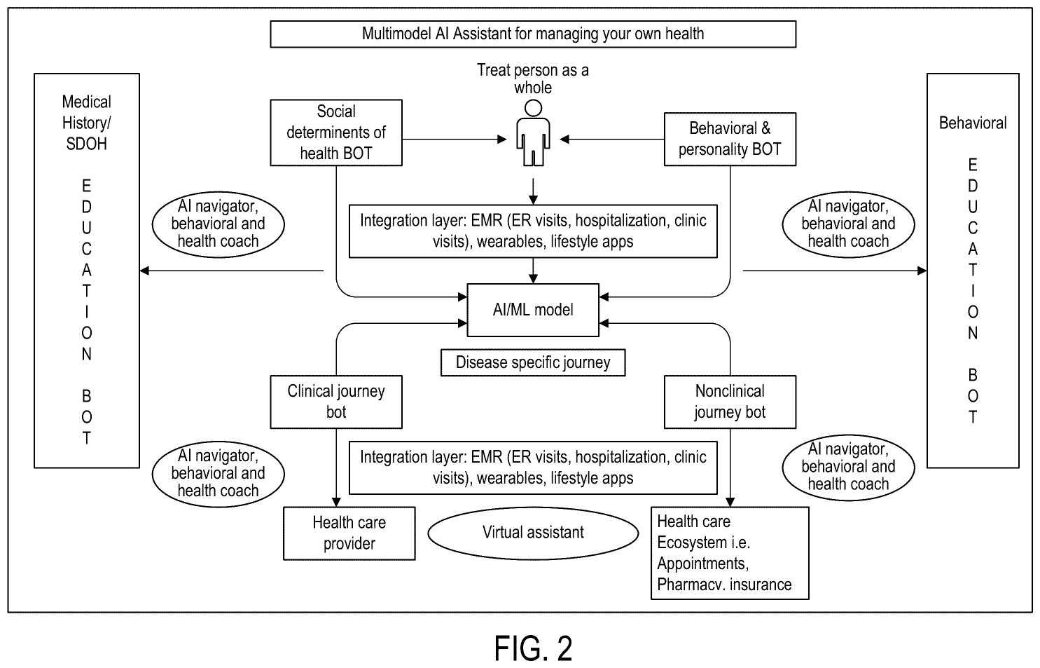

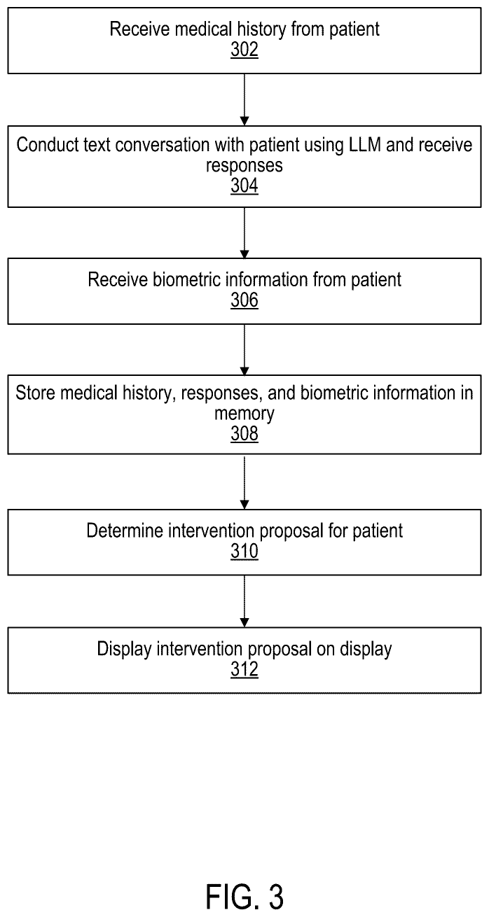

FIG. 1 illustrates a simplified block diagram of the computer system 101. As shown, the computer system 101 may comprise a processor 102 that is coupled to a random access memory (RAM) 104 and a non-transitory computer-readable memory 106 to implement embodiments described herein. For example, the processor may execute program instructions stored on the non-transitory memory to perform the method steps described herein, e.g., in reference to FIGS. 2-3. The non-transitory memory may have software programs stored thereon to implement the described embodiments, and the software programs may include artificial intelligence software, machine learning software and/or large language models. The computer system 101 may also comprise an input device 112 for receiving user input (e.g., a keyboard, mouse, touchpad, audio/visual input etc.) and a display device 110 for presenting output on a display. The computer 101 may also comprise an Input/Output (I/O) interface 108 that is coupled to the controller 102, the internet, or a wireless connection to a wireless local area network and/or a cellular network to receive input and/or information and provide output to facilitate the provision of healthcare to a patient, according to various embodiments. The computer system may be a personal computer (PC), laptop, UE device, or wearable device, in various embodiments.

### Atrial Fibrillation

In a normal heart rhythm, electrical signals start in the sinoatrial (SA) node and travel through the atria. The atria contract and bloods flows into the ventricles. The signal reaches the atrioventricular (AV) node, travels to the ventricles, and the ventricles contract. When an individual is suffering from atrial fibrillation (AF), disorganized electrical signals in the atria transmit chaotic impulses to the AV node, producing an irregular heartbeat. Due to the irregularity, blood may pool in the atria and form clots. AF is a common heart rhythm disorder amongst adults. It is a common cause of hospital admissions, as a chronic condition it is commonly recurrent leading to hospital readmissions and high treatment costs. 2.7 to 6.1 million Americans are affected by AF as of 2023, and some projections predict over 10 million Americans may suffer from AF by 2050. The costs of treating AF are large, with direct costs such as hospitalizations, medications and procedures, as well as indirect costs such as lost productivity and disability. For example, effective treatment of AF may involve symptom management to improve the patient's quality of life (e.g., to manage fatigue, palpitations, shortness of breath, etc.), maintenance of a normal heart rhythm through medication (e.g., antiarrhythmic medications) and/or ablation therapy, management of stroke risk, and risk factor management, among other possibilities.

### Digitally Assisted Healthcare

It may be desirable for patients with AF or other health conditions to receive ongoing education about the condition, guidance on treatment options, and answers to questions tailored to disease stage, progression, personal perception and co-morbidities. Many currently available health apps and digital health solutions are not tailored to the immediate health concerns for the patient, and a patient may undergo significant research to educate him/herself and go through a multi-step process to make their appointments and get adequate healthcare. Embodiments herein improve on these solutions by providing a hybrid model between a consumer-oriented digital solution, digital health via telemedicine, chronic condition management, remote physiologic monitoring, and value-based care for insurers and accountable care organizations.

Embodiments herein describe systems and methods to provide a versatile artificial intelligence (AI)-enabled digital health software that provide assistance to patients diagnosed with AF. Embodiments herein describe software that is executed by a computing device to function as a medical interface device and a direct-to-consumer digital tool to provide patients with AF comprehensive education information, medical history summary, and detailed description of treatment pathway options based on symptoms, co-morbidities, and other factors. While some embodiments are described in the context of treating AF, it is also within the scope of this disclosure to utilize the described methods for treating any of a variety of other adverse health conditions.

In some embodiments, the software is machine-learning AI software that is configured to self-learn and evolve into an augmented digital assistant that may improve patient outcomes and also improve the workflow of medical assistants, physician assistants, nurse practitioners, primary care providers, and specialists.

In some embodiments, the described methods may decrease patients' anxiety, improve timeliness and access to care, influence behavior with appropriate nudges that affect the outcomes of the disease and treatment, and reduce cost without adversely affecting safety and efficacy. Some embodiments may also help decrease health disparity and assist to overcome common barriers to care.

In some embodiments, software is configured to function as a history taking medical bot trained on machine learning software using large language models to generate an automated history from a conversation with a patient about his/her health. As used herein, the term “bot” refers to a set of services provided through a software program to provide a particular functionality. For example, an educational bot may interface with a patient through a UI to provide medical education on relevant health topics, a medical history bot may interface with the patient to ask questions and acquire a medical history, etc. In some embodiments, the chatbot generates a structured summary for review by a healthcare provider.

In some embodiments, a patient may be educated on risk factor modification for their health condition(s) based on the patient's individual risk factors, and further provided with treatment goals and options. In some embodiments, an interactive discussion is provided regarding treatment options, clinical decision making, and troubleshooting for recurrence of the health condition, exacerbation of symptoms, and/or other complications.

Embodiments herein provide a comprehensive platform for a patient to manage their own health to improve health outcomes. Some embodiments leverage generative artificial intelligence and data-driven insights using machine learning models to enhance patient care, facilitate communication between healthcare providers and patients, and empower individuals to take control of their health condition(s). By offering personalized care plans, remote monitoring, and access to educational resources, patients may benefit from more timely care, reduced health-related complications, reduced hospitalizations, and improved patient outcomes.

Some described embodiments provide educational content to fill knowledge gaps on health conditions for patients, utilize AI/ML models with patient input in a feedback loop for expert monitoring of the patient's condition, create medical transcripts and long-term plans for improving a patient's health and wellness, create actionable behavioral nudge units customized to each individual patient's needs using AI/ML models, and create actionable insights for reducing individual risk factors using AI/ML models.

### FIG. 2—Multimodal AI Model for Managing Patient Health

FIG. 2 is a flow diagram illustrating operation of a multimodal artificial intelligence (AI) and/or machine learning (ML) assistant for a patient to manage their own health, according to some embodiments. As illustrated, multiple modes of artificial intelligence (AI) interact with a patient to provide holistic health care assistance to the patient that incorporates information from multiple domains. A first AI/ML bot may be used to determine a medical history and social determinants of health for a patient, and a second AI/ML bot may be used to determine behavioral and personality aspects of the patient. These determined aspects may be fed into an AI/ML model that also receives the patients electronic medical records (EMR), biometric data from wearable devices, and/or information from lifestyle apps in order to determine a health intervention proposal to display for the patient. The AI/ML model may also interface with a health care provider and other entities in the healthcare system, to schedule appointments, provide potential medical diagnoses, receive care schedules, make appointments, file with insurance, and/or call prescriptions into pharmacies, among other possibilities.

### FIG. 3—Method of Providing a Health Intervention Proposal

FIG. 3 illustrates an example simplified block diagram of a method for determining a providing a health intervention proposal to a patient, according to some embodiments. Aspects of the method of FIG. 3 may be implemented by a system, such as illustrated in and described with respect to FIG. 1, among other systems and devices, as desired. For example, in some embodiments a non-transitory computer-readable memory medium stores program instructions which, when executed by a processor, cause a computing device such as a personal computer, a UE device, or a wearable device to perform the described method steps. In various embodiments, some of the elements of the methods shown may be performed concurrently, in a different order than shown, may be substituted for by other method elements, or may be omitted. Additional method elements may also be performed as desired. As shown, the method may operate as follows.

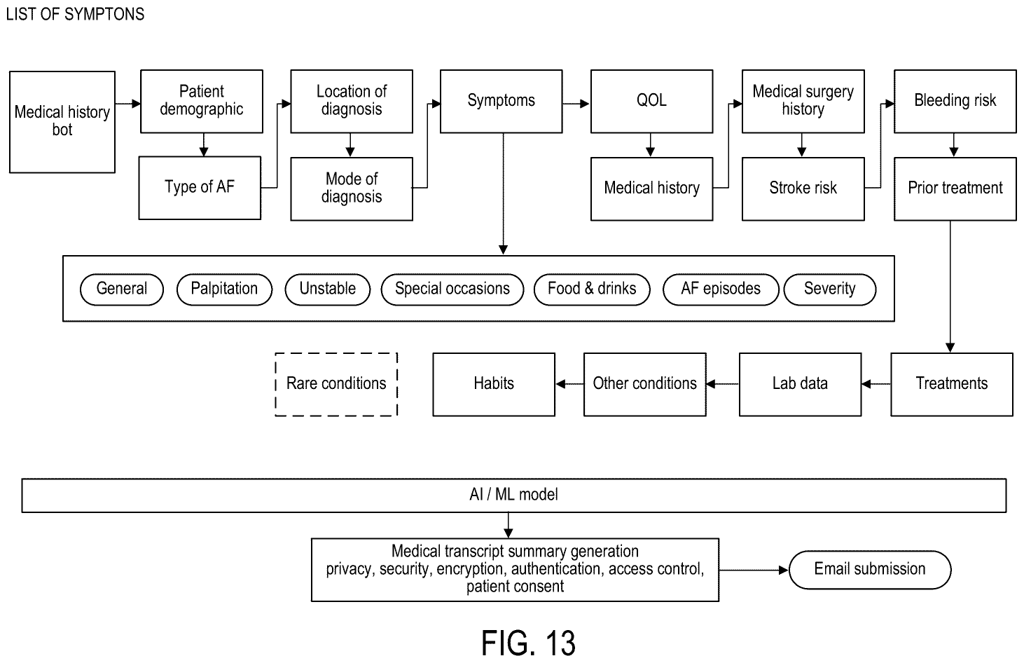

At 302, a medical history for a patient is received. In some embodiments, the medical history may be received as an electronic medical record (EMR) from a healthcare provider through a wired or wireless means (e.g., over the internet or a cellular network). Alternatively, in some embodiments a large language model (LLM) is configured to conduct a conversation with a patient over a user interface (UI), and the medical history is constructed by the processor (e.g., using machine learning software) based on the patient's responses in the conversation. For example, the LLM may ask the patient a series of questions related to the patient's health, similar to a series of questions that may be asked by a physician during an office visit, and the medical history may be constructed based on the patient's responses. The LLM may provide questions to further clarify the patient's responses and improve the accuracy and/or detail of the medical history, in some embodiments. In some embodiments, the medical history is combined with other health information related to the patient to generate a medical transcript summary. A detailed flowchart describing a method for generating a medical transcript for a patient is shown in FIG. 13.

In some embodiments, a care gap may be identified based on the medical history, and an indication of a treatment may be automatically displayed to the patient on a display based on the care gap. For example, the medical history may be analyzed to determine that any health conditions identified by the medical history have been followed up with appropriate treatment, based on current standards of care. Any discrepancy between the patient's treatment history and the standard of care for the patient's health condition(s) may be identified as a care gap, and a recommended treatment for these health condition(s) may be displayed to the patient.

At 304, a text conversation is conducted with the patient through a UI and using a LLM to obtain a plurality of responses. In some embodiments, conducting the text conversation includes displaying one or more questions to the patient on a display. The text conversation may be used to obtain additional information regarding the patient, that may be used to determine a health intervention proposal for the patient.

In some embodiments, the questions may be related to the patient's socioeconomic status, race, sex, gender, ethnicity, living environment, family status, education level, and/or healthcare access. These questions may be used to identify one or more social determinants of health for the patient. As used herein, social determinants of health (SDOH) are economic and social conditions that influence individual and group differences in health status. SDOH are health promoting factors found in one's living and working conditions (such as the distribution of income, wealth, influence, and power), rather than individual risk factors (such as behavioral risk factors or genetics) that influence the risk or vulnerability for a disease or injury. The identified SDOH may be subsequently utilized by a machine learning software to identify a potential medical diagnosis and/or an intervention proposal that is statistically likely to be effective for the patient, given their SDOH status. For example, certain demographic characteristics may be statistically associated with a particular medical diagnosis, genetic condition, behavioral tendency, health risk factor, or another factor relevant to the patient's health. This statistical association may be utilized to provide an intervention proposal that is more likely to be relevant and effective in improving a patient's health, as described in greater detail below.

In some embodiments, the one or more questions inquire regarding personal aspects of a patient, such as their behavioral habits, personality, emotional state, attitudes toward a disease, willingness to modify behavior, and/or lack of education regarding one or more health conditions identified in their medical history. Responses to these questions may likewise be utilized by machine learning software to identify an intervention proposal with which the patient may be more likely to comply. For example, an identified reluctance of a patient to modify behavior to address a health condition may be used to select a less obtrusive intervention proposal, as described in greater detail below.

In some embodiments, separate artificial intelligence modules may be used to determine the social determinants of health and the personal aspects of the patient. The two AI modules may both utilize the same UI to ask questions of the patient, but they may operate according to separate machine learning algorithms, with their own sets of parameters, weights, etc.

At 306, biometric information for the patient is received from at least one biometric device. The biometric device may include a smart watch, a blood pressure monitor, a heart rate monitor, a weight scale, an activity tracker, and/or a digital health app that is specifically tailored to atrial fibrillation (AF) or another health condition, among other possibilities. The biometric information may describe a current biometric measurement of the patient, or it may describe a biometric history over some period of time, as measured by the biometric device.

At 308, the medical history, the plurality of responses, and/or the biometric information are stored in the non-transitory computer-readable memory medium. The medical history, the plurality of responses, and/or the biometric information may be subsequently accessed by the processor, for processing and/or for provision to a health provider or facility.

At 310, an intervention proposal is determined using a machine learning software based at least in part on the medical history, the plurality of responses, and the biometric information. The intervention proposal may take a variety of forms, in different embodiments. For example, the intervention proposal may be a behavioral “nudge”, meaning a suggestive behavior modification that may help improve a health condition of the patient. The behavioral nudge may be determined not only from the health condition that is intended to be treated, but also from various personal aspects of the patient's personality profile, demographics, behavioral habits, medical history, and biomedical information. In some embodiments, the intervention proposal is a personalized action item designed to help improve the patient's health outcomes.

In some embodiments, health or lifestyle information is also received from one or more health software applications or lifestyle apps operated by the patient, and the intervention proposal is determined further based on the health or lifestyle information. For example, the patient may operate one or more health apps on the same or a different device than the device that is determining the intervention proposal. The processor may be configured to interface with the one or more health apps, automatically or responsive to user input, to receive health information collected by the app(s).

In some embodiments, determining the intervention proposal includes determining one or more potential medical diagnoses for the patient from the medical history and the biometric information. The potential medical diagnoses may include AF and/or other health conditions, in some embodiments. The potential medical diagnoses may be provided to a medical call center or a health care provider (e.g., over a wired or wireless connection through a cellular network and/or an internet protocol network). A care schedule to provide to the patient may be received from the medical call center or health care provider responsive to providing the potential medical diagnoses. The potential medical diagnoses may be determined by machine learning software and may list one or more likely health conditions suffered by the patient. This information may be used by the medical call center or health care provider to facilitate their determination of a medical diagnosis, to expedite their development of a care schedule to treat the patient. The care schedule may be displayed to the patient on a display.

Additionally or alternatively, in some embodiments, the intervention proposal may be determined from the care schedule. For example, the machine learning software may receive the care schedule, and may determine an intervention proposal that takes into account not only the care schedule but also personal behavioral traits of the patient. Advantageously, the intervention proposal may then be tailored to the personality and other traits of the patients, to increase the likelihood of treatment compliance by the patient.

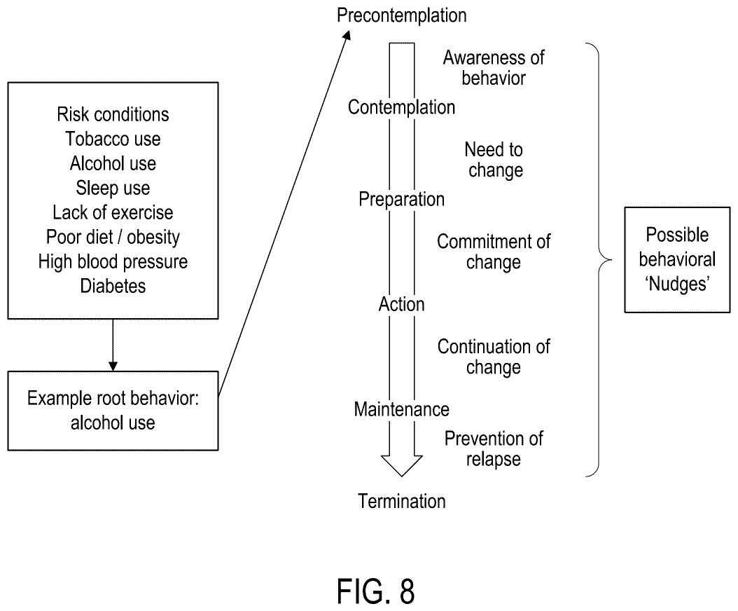

In some embodiments, determining the intervention proposal includes determining a root behavioral cause for a medical issue experienced by the patient from a plurality of risk conditions. FIG. 8 illustrates aspects of these embodiments. In some embodiments, the risk conditions may include tobacco use, alcohol use, sleep issues, lack of exercise, poor diet, obesity, hypertension, and/or diabetes, among other possibilities. The root behavioral cause may be determined based on the medical history, the plurality of responses, and/or the biometric information. For example, the text conversation conducted by the LLM may be designed to probe the patient to determine a likely root behavior that is a primary cause of a health condition identified by the medical history and/or by the biometric information. As one specific example, if a patient is suffering from a metabolic health disorder, the LLM may ask questions of the patient to determine whether the metabolic health disorder is more likely to be caused by alcohol consumption, poor diet, excess tobacco use, stress, lack of exercise, or another factor or combination of factors.

The method may continue by conducting a follow-up text conversation with the patient based on the identified root behavioral cause. The follow-up text conversation may ask the patient questions to determine a level of awareness and/or a willingness to intervene for the patient in regard to the root behavioral cause. The determined level of awareness may be one of ignorance, precontemplation, or contemplation of how the root behavioral cause may be contributing to the patient's health condition, in some embodiments. The determined willingness to intervene may be an unwillingness, preparation, action, or maintenance, among other possibilities. The intervention proposal may then be determined based on the root behavioral cause, the level of awareness, and/or the willingness to improve. For example, the follow-up conversation may ask questions to determine how aware the patient is of the root behavioral cause of their health condition, and/or how willing they are to take active steps to modify their behavior. The intervention proposal may then be tailored to suit the patient's current state of mind, in addition to being selected to effectively treat the health condition. For example, if it is determined that a patient is unaware that the root behavioral cause may be causing their health condition, the intervention proposal may simply be to inform the patient that they are conducting the behavior and that the behavior has been shown to cause their health condition. If it is determined that the patient is aware that the root behavioral cause may be causing their health condition, the intervention proposal may suggest that changing the behavior may result in improved health outcomes, and may present one or more specific behavior modifications to provide this benefit. If it is determined that the patient is currently undertaking steps to address the root behavioral cause, the intervention proposal may offer advice on maintaining the desired change in behavior and/or preventing behavioral relapse.

At 312, the intervention proposal is displayed on a display.

In some embodiments, the method may further include receiving a response from the patient to the intervention proposal. For example, the patient may indicate in a response how willing they are to incorporate the intervention proposal. The method described in FIG. 3 may then iterate based on the patient response, updating the personal aspects of the patient stored in memory and determining an updated intervention proposal, for example. For example, the personalized digital healthcare assistant may implement a feedback process, where patient responses to the displayed intervention proposal are used to follow the patient along in their treatment plan, providing updated intervention proposals as the patient's treatment progresses.

For example, in some embodiments, after displaying the intervention proposal on the display, a second text conversation is conducted with the patient using the UI to determine a degree of compliance with the intervention proposal. The behavior profile of the patient may be modified based at least in part on the degree of compliance with the intervention proposal. A second intervention proposal may be determined based at least in part on the degree of compliance and the modified behavior profile the second intervention proposal may be displayed on the display. For example, if a patient was not compliant with the original intervention proposal, the behavior profile may be modified to indicate this reluctance to comply, and the second intervention proposal may present a less significant or difficult behavioral modification to address the patient's health condition. Alternatively, if the patient was highly compliant, the behavior profile may be modified to indicate the high level of compliance, and the second intervention proposal may be a more significant behavioral modification, or a next step in a treatment plan for the patient.

In some embodiments, the UI may be configured to receive user input to interface with a medical cell center and/or health provider, e.g., to schedule an appointment or procedure associated with the intervention proposal. In some embodiments, the UI may be configured to receive user input in response to the displayed intervention proposal, and may be configured to interface with another health app of the patient to modify parameters of the health app.

In some embodiments, the method further includes displaying first and second health prognoses on the display based on following and not following the intervention proposal, respectively. Advantageously, this may increase patient compliance by showing the patient a desirable health outcome if the intervention proposal is followed, and/or an undesirable health outcome if the intervention proposal is not followed.

### Additional Description

The following numbered paragraphs provide additional information related to technical aspects of various embodiments.

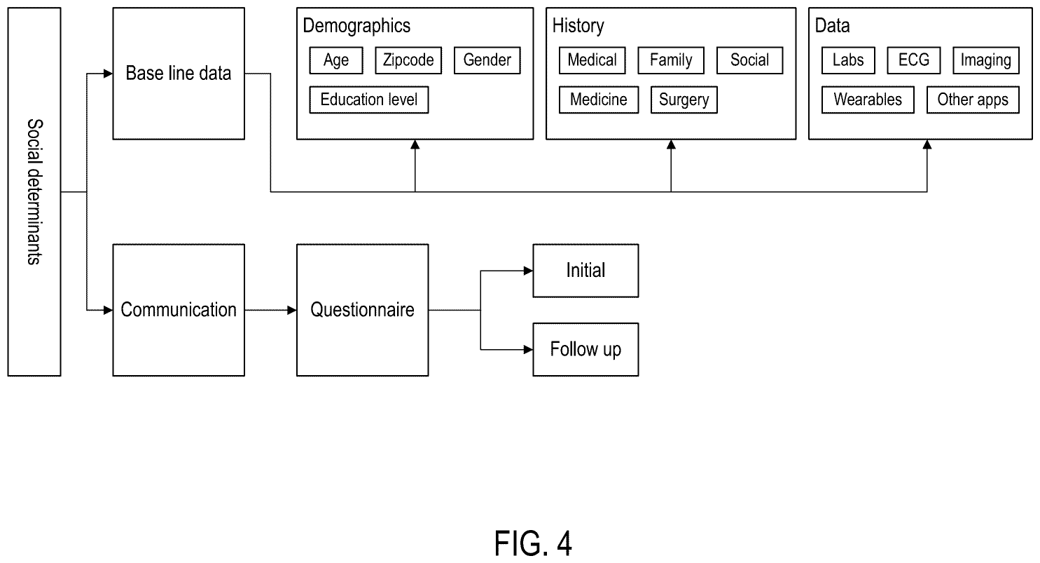

FIG. 4 is a diagram illustrating factors that may contribute to social determinants of health (SDOH), according to some embodiments. baseline data may be acquired for the patient, which may include demographic data such as age, zipcode, gender and education level, medical history data including EMR data, family history, social information, medicines current taken by the patient, and previous and scheduled surgical procedures. Data from lab tests, electrocardiograms (ECGs), imaging data, and biometric or health data from wearable devices and other apps may also be used as base line data to determine the SDOH for the patient. The AI/ML model may then communicate with the patient with an initial questionnaire and/or follow-up questions to further refine the determined SDOH.

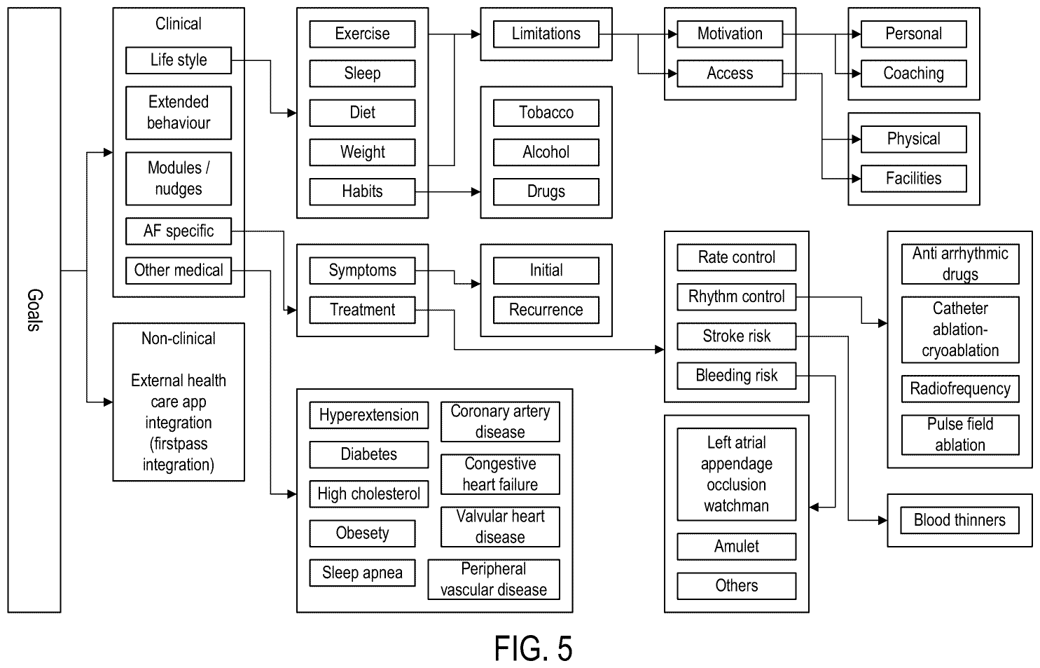

FIG. 5 is a diagram illustrating examples of clinical and non-clinical health goals, according to some embodiments. The goals may apply to a variety of domains, such as patient lifestyle, extended behavior, health condition-specific goals (e.g., AF-specific goals), and non-clinical goals. The goals may be organized and stored as a hierarchical data structure, with multiple stages of subcategories for each goal category. The goals may be displayed to the patient in association with an intervention proposal, in some embodiments.

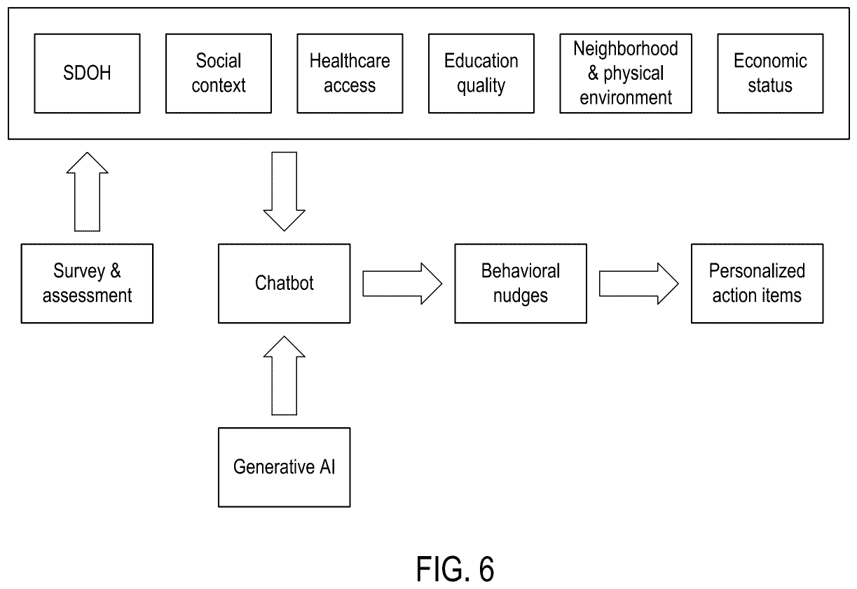

FIG. 6 is a flowchart illustrating the generation of personalized action items for a patient, according to some embodiments. A chatbot may receive information related to both the SDOH and behavioral and personality aspects of the patient from a generative AI model. The chatbot may have a conversation with the patient to propose behavioral nudges and personalized action items to assist the patient in improving their health conditions, in some embodiments.

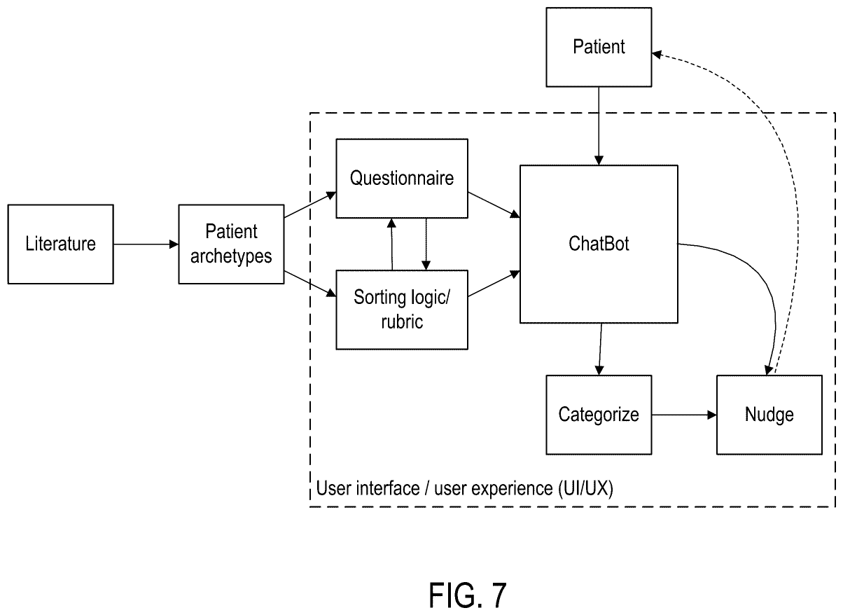

FIG. 7 is a flowchart illustrating a user interface for categorizing patient archetypes and advising patient behavior, according to some embodiments. As illustrated, a plurality of patient archetypes may be determined by an AI/ML model based on the medical literature. As used herein, a patient archetype refers to a particular grouping of behavioral and/or personality traits. For example, a patient may be classified as a patient archetype that tends to be trusting of and compliant to physician instructions. Alternatively, a patient may be classified as an archetype that is reluctant to modify his/her behavior or otherwise follow health advice. The LLM may converse with the patient with a questionnaire, and may process the responses using sorting logic and/or rubrics to categorize the patient with a particular patient archetype. Based on the patient archetype of the patient, the AI/ML model may determine a behavioral nudge to display for the patient as an intervention proposal, where the behavioral nudge may be selected based on statistical tendencies of the patient archetype of the patient. This process may be iterated, as responses from the patient to the proposed behavioral nudge may be received and processed to further customize a subsequent intervention proposal.

FIG. 8 is a diagram illustrating the determination of a possible behavioral nudge based on a root behavior and a patient's perspective, according to some embodiments. As illustrated, alcohol use is identified as a root behavioral cause of a patient's health condition. It is then determined, through conversing with the patient with an LLM, how aware the patient is of his/her excess alcohol use, and how willing the patient is to modify his/her alcohol consumption. A behavioral nudge is selected to display for the patient based on the determined mental state of the patient, and their likelihood to comply with various proposed behavioral changes.

In some embodiments, user personas are determined for a patient using a classification algorithm that utilizes feature vectors derived from patient health data received through the app, web, as well as any data collected from the EMR, wearables, doctor's notes, lab results, and digital image data. User personas may be defined for groups of patients that share a particular cluster of attributes or characteristics. Any inputs for the clustering algorithms may be used directly or after preprocessing the data to extract derived quantities ranging from statistical means of central tendency or parameters derived from time series analyses. In some embodiments, the feature vectors are periodically updated and used to determine the specific persona a user belongs to and also used to move the user to a different persona, if necessary, when new data arrives.

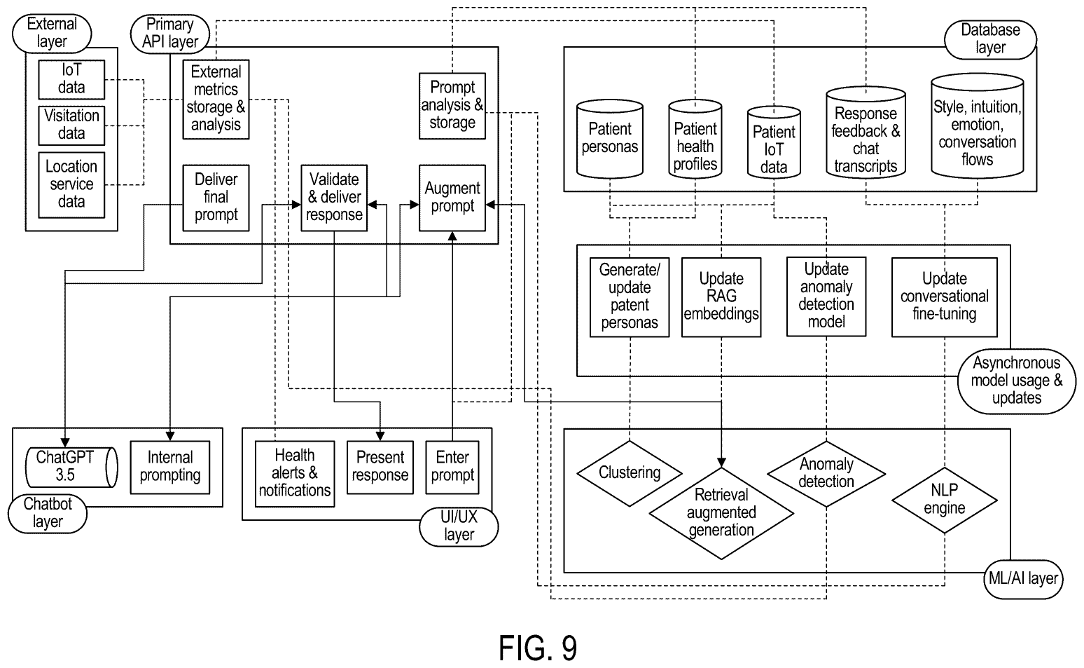

FIG. 9 is a diagram illustrating different computational modules for providing health services to a patient, according to some embodiments. In some embodiments, an education bot provides personalized and targeted education materials to each user (patient). The education material can be in multiple media formats such as pdf, docx, img, video, audio etc. Personalization of specific documents recommended to each user may be based on a custom algorithm that determines documents to recommend based on past documents viewed, time spent, user feedback, etc. The recommend document(s) may be determined not just on feedback received from that specific user, but also from feedback from a group of users that share a persona of the user.

The recommendation may be determined either in real-time or through a batch process, which is triggered each time any user's (with that particular persona) viewing history crosses a preset threshold.

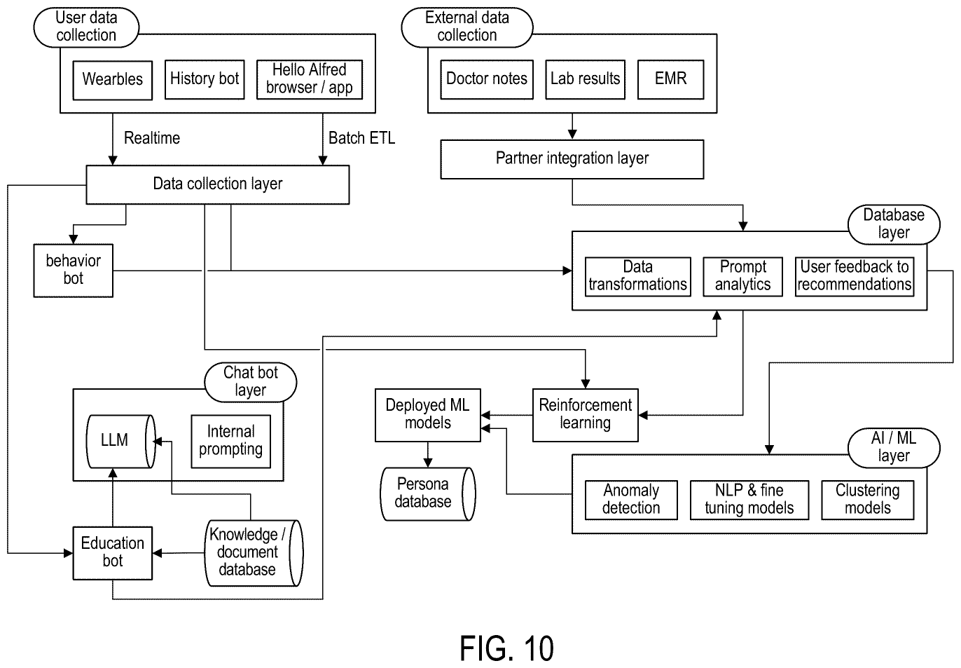

FIG. 10 is a diagram illustrating data collection and AI bot operation for providing health services to a patient, according to some embodiments. Persona determination/clustering:

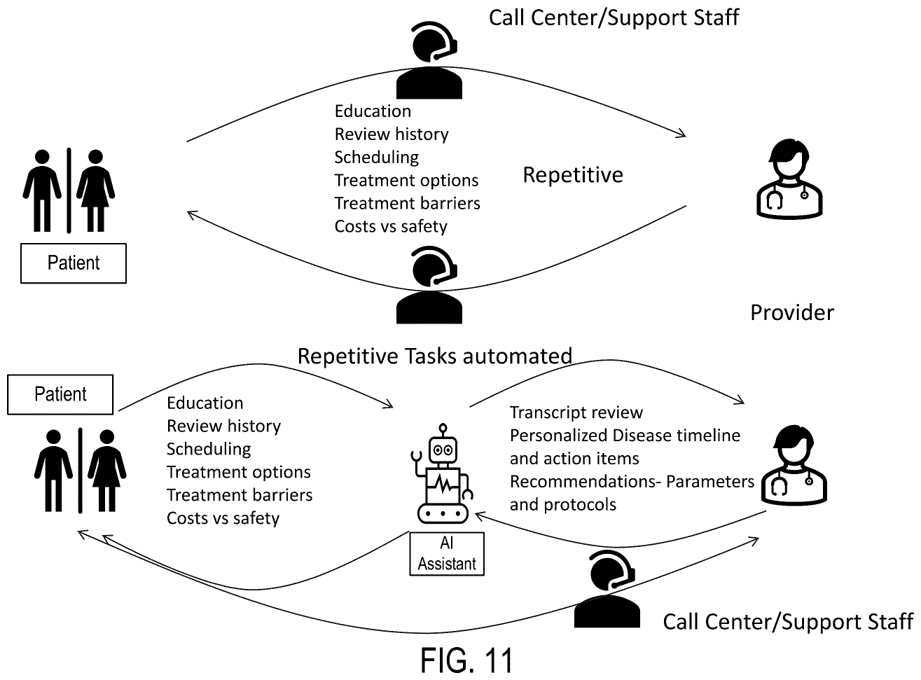

FIG. 11 contrasts the provision of healthcare to a patient with and without a multimodal AI model, according to some embodiments. The top half of FIG. 11 illustrates a repetitive and laborious process of a patient communicating with a call center and/or healthcare support staff as an intermediary to a healthcare provider to obtain education related to their health condition(s), review medical history, schedule appointments, discuss treatment options and barriers, and discuss costs and safety of various treatment options. In contrast, the bottom half of FIG. 11 illustrates a more efficient process where an AI assistant serves as an intermediary between the healthcare provider and/or the call center/support staff. The AI assistant autonomously performs patient education, medical history review, scheduling, and discussion of treatment options, barriers, costs, and safety with the patient. The AI assistant further interfaces with the healthcare provider to provide transcript review of its conversations with the patient, a personalized disease timeline and action items, and recommended parameters and protocols for treating the patient's health condition(s).

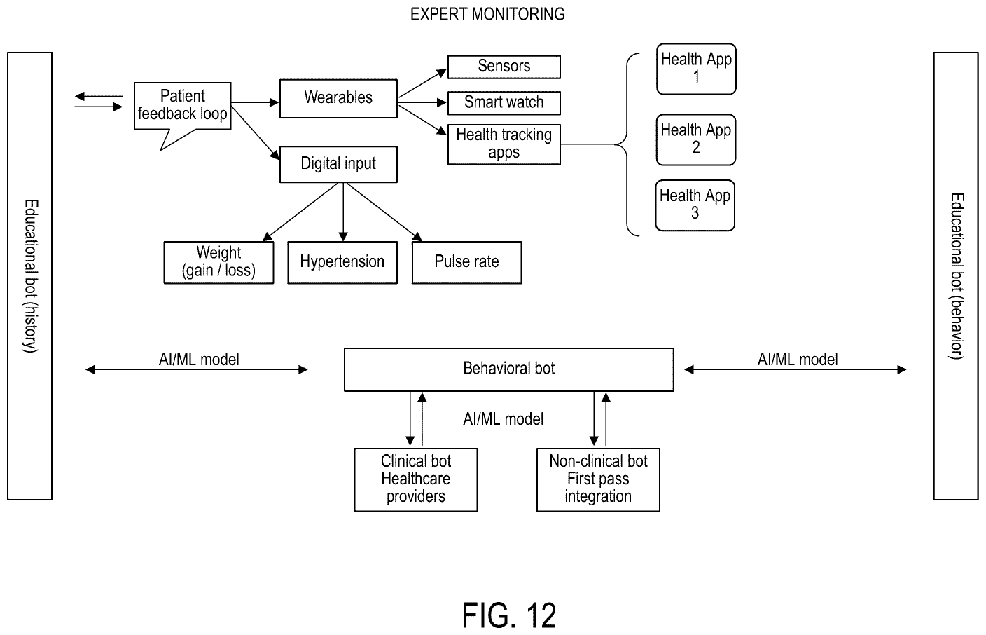

FIG. 12 is a flowchart showing a computational flow for providing expert health monitoring for a patient, according to some embodiments. As illustrated, a feedback loop is operated between a medical history educational bot and biometric measurements from wearable devices.

FIG. 13 is a flowchart illustrating a method for a medical history bot to construct a medical history for a patient with AF, according to some embodiments. The method starts by identifying demographic information for the patient. It is then determined what type of AF the patient may have. The location and mode of the diagnosis is determined, and the text conversation is utilized to determine symptoms that the patient is experiencing. The text conversation may present a list of potential symptoms to the patient (e.g., general malaise, palpitations, feeling unstable, intermittent symptoms on special occasions, issues tolerating food and drinks, episodes of AF, and an indication of severity), and the patient may select one or more symptoms from the list. The bot may then determine a quality of life (QOL) of the patient, and establish a medical and surgical history. The bot may then determine a level of stroke risk and bleeding risk for the patient, determine whether the patient has had prior or ongoing treatments, whether the patient has received lab results. The bot may also inquire about other conditions of the patient, and their habits. Optionally, the bot may inquire regarding one or more rare conditions, if the acquired data indicates that the rare conditions may be likely for the patient. This collected information may then be processed by an AI/ML model to generated a medical transcript summary. The medical transcript may be provided, e.g., through secure email, to a healthcare provider.

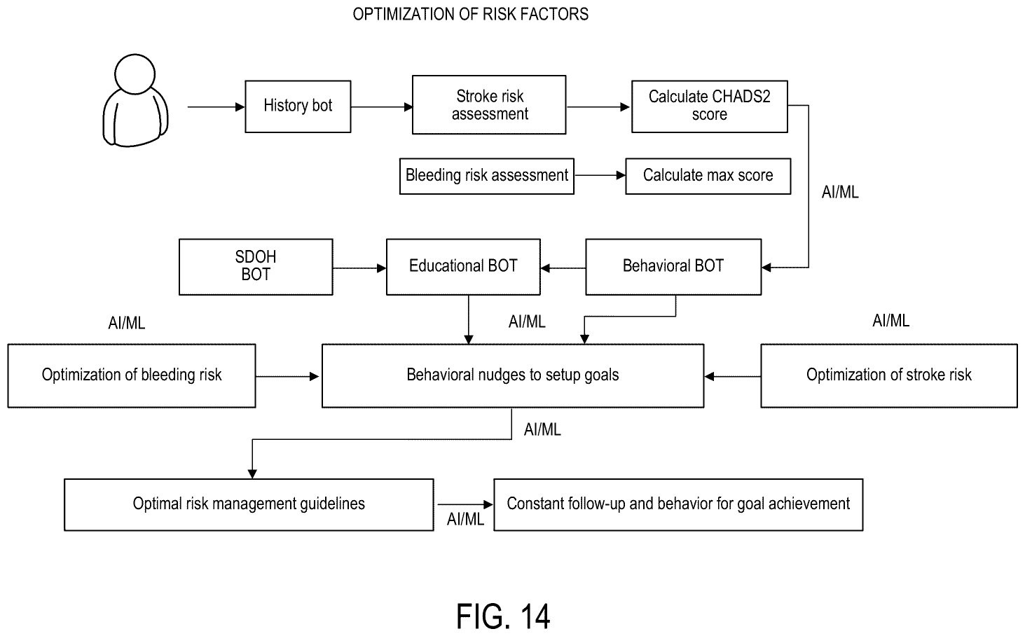

FIG. 14 is a flowchart illustrating a workflow for optimizing risk factors and providing behavioral nudges to a patient, according to some embodiments. As illustrated medical history of the patient obtained from a history bot may be used to assess a quantitative level of stroke risk, and/or a quantitative level of bleeding risk for the patient. A social determinant of health (SDOH) bot and a behavioral bot may provide information to an educational bot, and the educational bot may use this information to propose one or more behavioral nudges for the patient. The behavioral nudges may be determined from suggested nudges received from the educational bot, as well as from the quantitative estimates of bleeding and stroke risk. For example, higher risk levels may be used to select more significant behavioral changes. Constant follow-up and behavioral analysis may be performed to achieve desired health goals.

Embodiments of the present disclosure may be realized in any of various forms. For example, some embodiments may be realized as a computer-implemented method, a computer-readable memory medium, or a computer system. Other embodiments may be realized using one or more custom-designed hardware devices such as ASICs. Still other embodiments may be realized using one or more programmable hardware elements such as FPGAs.

In some embodiments, a non-transitory computer-readable memory medium may be configured so that it stores program instructions and/or data, where the program instructions, if executed by a computer system, cause the computer system to perform a method, e.g., any of the method embodiments described herein, or, any combination of the method embodiments described herein, or, any subset of any of the method embodiments described herein, or, any combination of such subsets.

In some embodiments, a device may be configured to include a processor (or a set of processors) and a memory medium, where the memory medium stores program instructions, where the processor is configured to read and execute the program instructions from the memory medium, where the program instructions are executable to implement any of the various method embodiments described herein (or, any combination of the method embodiments described herein, or, any subset of any of the method embodiments described herein, or, any combination of such subsets). The device may be realized in any of various forms.

Although the embodiments above have been described in considerable detail, numerous variations and modifications will become apparent to those skilled in the art once the above disclosure is fully appreciated. It is intended that the following claims be interpreted to embrace all such variations and modifications.

What is claimed is:

1. A non-transitory computer-readable memory medium comprising program instructions which, when executed by a processor, cause a computing device to:
receive a medical history for a patient;
conduct, using a large language model (LLM), a text conversation with the patient using a user interface (UI) to obtain a plurality of responses;
receive biometric information for the patient from at least one biometric device;
store the medical history, the plurality of responses, and the biometric information in the non-transitory computer-readable memory medium;
determine, using a machine learning software, an intervention proposal based at least in part on the medical history, the plurality of responses, and the biometric information; and
display the intervention proposal on a display.

2. The non-transitory computer-readable memory medium of claim 1,
wherein, in determining the intervention proposal, the program instructions are further executable to cause the computing device to:
determine one or more potential medical diagnoses for the patient based at least in part on the medical history and the biometric information;
provide the one or more potential medical diagnoses to a medical call center;
receive a care schedule from the medical call center to provide to the patient; and
determine the intervention proposal further based at least in part on the care schedule.

3. The non-transitory computer-readable memory medium of claim 2,
wherein the one or more potential medical diagnoses comprise a diagnosis of atrial fibrillation.

4. The non-transitory computer-readable memory medium of claim 1, wherein the program instructions are further executable to cause the computing device to:
identify at least one care gap based at least in part on the medical history;
automatically display, to the patient on the display, an indication of a treatment based at least in part on the at least one care gap.

5. The non-transitory computer-readable memory medium of claim 1, wherein the program instructions are further executable to cause the computing device to:
display a first health prognosis on the display based on following the intervention proposal; and
display a second health prognosis on the display based on not following the intervention proposal.

6. The non-transitory computer-readable memory medium of claim 1, wherein the program instructions are further executable to cause the computing device to:
receive health information from one or more health software applications operated by the patient,
wherein the intervention proposal is determined further based at least in part on the health information.

7. The non-transitory computer-readable memory medium of claim 1,
wherein conducting the text conversation comprises displaying one or more questions to the patient on the display,
wherein the one or more questions are related to one or more of the patient's socioeconomic status, race, sex, gender, ethnicity, living environment, behavioral habits, family status, education level, personality, emotional state, and healthcare access.

8. The non-transitory computer-readable memory medium of claim 1,
wherein conducting the text conversation comprises displaying one or more questions to the patient on a display, wherein the one or more questions inquire regarding one or more of:
social determinants of health for the patient;
a personality of the patient;
an attitude toward disease for the patient;
a willingness to modify behavior of the patient;
a medical history of the patient; and
an education gap of the patient.

9. The non-transitory computer-readable memory medium of claim 1,
wherein, in determining the intervention proposal based at least in part on the medical history, the plurality of responses, and the biometric information, the program instructions are executable by the processor to cause the computing device to:
determine, from a plurality of risk conditions, a root behavioral cause for a medical issue experienced by the patient based on one or more of the medical history, the plurality of responses, and the biometric information;
conduct a second text conversation with the patient based at least in part on the root behavioral cause, wherein the second text conversation asks the patient questions to determine a level of awareness and/or a willingness to intervene for the patient in regard to the root behavioral cause; and
determine the intervention proposal based at least in part on the root behavioral cause, the level of awareness, and/or the willingness to improve.

10. The non-transitory computer-readable memory medium of claim 9,
wherein the plurality of risk conditions comprise one or more of:
tobacco use;
alcohol use;
sleep issues;
lack of exercise;
poor diet and/or obesity;
hypertension; and
diabetes,
wherein the level of awareness comprises one of:
ignorance;
precontemplation; and
contemplation; and
wherein the willingness to intervene comprises one of:
unwillingness;
preparation;
action; and
maintenance.

11. The non-transitory computer-readable memory medium of claim 1,
wherein, in determining the intervention proposal based at least in part on the medical history, the plurality of responses, and the biometric information, the machine learning software is configured to:
determine, using a first artificial intelligence module, one or more social determinants of health of the patient based on the plurality of responses; and
determine, using a second artificial intelligence module, a personality profile of the patient based on the plurality of responses,
wherein the intervention proposal is determined further based on one or both of the social determinants of health and the personality profile.

12. The non-transitory computer-readable memory medium of claim 1, wherein the program instructions are further executable to cause the computing device to:
after displaying the intervention proposal on the display, conduct a second text conversation with the patient using the UI to determine a degree of compliance with the intervention proposal;
modify a behavior profile of the patient based at least in part on the degree of compliance with the intervention proposal;
determine a second intervention proposal based at least in part on the degree of compliance and the modified behavior profile; and
display the second intervention proposal on the display.

13. A method, comprising:
by a computing device:
receiving a medical history for a patient;
conducting, using a large language model (LLM), a text conversation with the patient using a user interface (UI) to obtain a plurality of responses;
receiving biometric information for the patient from at least one biometric device;
storing the medical history, the plurality of responses, and the biometric information in the non-transitory computer-readable memory medium;
determining, using a machine learning software, an intervention proposal based at least in part on the medical history, the plurality of responses, and the biometric information; and
displaying the intervention proposal on a display.

14. The method of claim 13,
wherein determining the intervention proposal comprises:
determining one or more potential medical diagnoses for the patient based at least in part on the medical history and the biometric information;
providing the one or more potential medical diagnoses to a medical call center;
receiving a care schedule from the medical call center to provide to the patient; and
determining the intervention proposal further based at least in part on the care schedule.

15. The method of claim 14,
wherein the one or more potential medical diagnoses comprise a diagnosis of atrial fibrillation.

16. The method of claim 13, the method further comprising:
identifying at least one care gap based at least in part on the medical history;
automatically displaying, to the patient on the display, an indication of a treatment based at least in part on the at least one care gap.

17. The method of claim 13,
wherein determining the intervention proposal based at least in part on the medical history, the plurality of responses, and the biometric information, the machine learning software comprises:
determining, using a first artificial intelligence module, one or more social determinants of health of the patient based on the plurality of responses; and
determining, using a second artificial intelligence module, a personality profile of the patient based on the plurality of responses,
wherein the intervention proposal is determined further based on one or both of the social determinants of health and the personality profile.

18. The method of claim 13, further comprising:
after displaying the intervention proposal on the display, conducting a second text conversation with the patient using the UI to determine a degree of compliance with the intervention proposal;
modifying a behavior profile of the patient based at least in part on the degree of compliance with the intervention proposal;
determining a second intervention proposal based at least in part on the degree of compliance and the modified behavior profile; and
displaying the second intervention proposal on the display.

19. A computing device, comprising:
one or more processors;
a non-transitory computer-readable memory medium coupled to the one or more processors; and
a display,
wherein the memory medium stores program instructions that are executable by the one or more processors to cause the computing device to:
receive a medical history for a patient;
conduct, using a large language model (LLM), a text conversation with the patient using a user interface (UI) to obtain a plurality of responses;
receive biometric information for the patient from at least one biometric device;
store the medical history, the plurality of responses, and the biometric information in the non-transitory computer-readable memory medium;
determine, using a machine learning software, an intervention proposal based at least in part on the medical history, the plurality of responses, and the biometric information; and
display the intervention proposal on a display.

20. The computing device of claim 19,
wherein, in determining the intervention proposal based at least in part on the medical history, the plurality of responses, and the biometric information, the program instructions are executable by the one or more processors to cause the computing device to:
determine, from a plurality of risk conditions, a root behavioral cause for a medical issue experienced by the patient based on one or more of the medical history, the plurality of responses, and the biometric information;
conduct a second text conversation with the patient based at least in part on the root behavioral cause, wherein the second text conversation asks the patient questions to determine a level of awareness and/or a willingness to intervene for the patient in regard to the root behavioral cause; and
determine the intervention proposal based at least in part on the root behavioral cause, the level of awareness, and/or the willingness to improve.
